(19) United States
(12) Patent Application Publication
(10) Pub. No.: US 2020/0239829 A1
(43) Pub. Date: Jul. 30, 2020

(54) DRY MATRIX FOR EMBEDDING VIABLE ESCHERICHIA COLI, METHOD OF MAKING SAME AND USE THEREOF

(71) Applicant: Prevtec Microbia Inc., Montreal (CA)

(72) Inventor: Eric Nadeau, Otterburn Parc (CA)

(73) Assignee: Prevtec Microbia Inc., Montreal (CA)

(21) Appl. No.: 16/850,919

(22) Filed: Apr. 16, 2020

Related U.S. Application Data
(63) Continuation of application No. 16/717,411, filed on Dec. 17, 2019.
Application No. 16/717,411 is a continuation of application No. 15/550,453, filed on Aug. 11, 2017.
(60) Provisional application No. 62/114,829, filed on Feb. 11, 2015.

(51) Int. Cl. C12N 1/04; C08L 3/02; C08K 11/00; A23K 20/10; C12N 11/10; C08L 5/02; C08K 5/1545; C08L 5/04; C12N 1/20

(52) U.S. Cl. CPC C12N 1/04; C08L 3/02; C08K 11/00; A23K 20/10; C12N 1/20; C08L 5/02; C08K 5/1545; C08L 5/04; C12N 11/10

(57) ABSTRACT

There is provided viable Escherichia coli (E. coli) embedded in a matrix, wherein said matrix has a water activity (a_{w})≤0.3, and wherein said matrix comprises a hydrocolloid-forming polysaccharide, a second polysaccharide which is different from the first polysaccharide, and a disaccharide which includes sucrose, trehalose, or a combination thereof. There is also provided methods of making same and use thereof.

### RELATED APPLICATIONS

The present application is a continuation of U.S. patent application Ser. No. 16/717,411, filed Dec. 17, 2019, which is a continuation of U.S. patent application Ser. No. 15/550,453, filed Aug. 11, 2017, which is a National Phase of International Application No. PCT/CA2016/050129, filed Feb. 11, 2016, which claims the benefit of U.S. Provisional Application No. 62/114,829, filed on Feb. 11, 2015 by Eric Nadeau. The contents of the above-referenced documents are incorporated herein by reference in their entirety.

### TECHNICAL FIELD

This application generally relates to the field of improved dry matrices for embedding viable E. coli, method of making same and use thereof.

### BACKGROUND

Bacterial spores are dormant life forms which can exist in a desiccated and dehydrated state indefinitely. For humans, bacterial spores are available either as over-the-counter prophylactics for mild gastrointestinal disorders, such as diarrhea, or as health foods or nutritional supplements. In the agricultural industry, bacterial spores are also receiving increasing attention as potential alternatives to antibiotics as growth promoters (Hong et al., FEMS Microbiology Reviews, 2005, 29: 813-835). Escherichia coli (E. coli) are, however non-spore-forming, and as such, are less resistant to desiccation and/or dehydration conditions than spore-forming bacteria. In many applications, it is nevertheless necessary to preserve and store E. coli bacteria in a form that affords sufficient viability and/or sufficient bacterial bioactivity for a given purpose.

In this regard, various practical preservation and storage conditions for bacteria have been previously suggested.

Freeze-drying (also named lyophilisation) is often used for preservation and storage of bacteria because of the low temperature exposure during drying (Rhodes, Exploitation of microorganisms ed. Jones, D G, 1993, p. 411-439, London: Chapman & Hall). However, it has the undesirable characteristics of significantly reducing viability as well as being time and energy-intensive. Protective agents have been proposed, but the protection afforded by a given additive during freeze-drying varies with the species of micro-organism (Font de Valdez et al., Cryobiology, 1983, 20: 560-566).

Air drying such as with desiccation has also been used for preservation and storage of bacteria. While vacuum drying is a similar process as freeze-drying, it takes place at 0°-40° C. for 30 min to a few hours. The advantages of this process are that the product is not frozen, so the energy consumption and the related economic impact are reduced. In the product point of view, the freezing damage is avoided. However, desiccation at low or ambient temperature is slow, requires extra precautions to avoid contamination, and often yields unsatisfactory viability (Lievense et al., Adv Biochem Eng Biotechnol., 1994, 51:71-89).

Encapsulating bacteria in hydrocolloid-forming polysaccharide matrix, such as Calcium-alginate (Ca-alginate) beads, has also been used for preservation and storage of bacteria in a broad and increasing range of different applications (Islam et al., J. Microbiol. Biotechnol., 2010, 20:1367-1377). To maintain the bacteria in a metabolically and physiologically competent state and thus obtain the desired benefit, it has been suggested to add to such matrices a suitable preservative formulation. Preservative formulations typically contain active ingredients in a suitable carrier and additives that aid in the stabilization and protection of the microbial cells during storage, transport and at the target zone.

Mannitol has been described as an effective preservative formulation component for Ca-alginate encapsulated bacteria during freeze-drying as it affords high bacterial viability up to 10 weeks under room temperature and water activity (a_{w}) of less than 0.2 (Efiuvwevwere et al., Appl. Microbiol. Biotechnol., 1999, 51:100-104). A synergistic mixture of trehalose and a sugar alcohol has also been described as an effective preservative formulation component for air-dried Ca-alginate encapsulated bacteria, where trehalose is used instead of sucrose for its significantly higher glass transition temperature, i.e., 110° C. vs. only 65° C., respectively (U.S. Pat. No. 8,097,245). A synergistic mixture of carboxylic acid salts and hydrolyzed proteins has also been described as an effective preservative formulation component for freeze-dried Ca-alginate encapsulated bacteria (U.S. 2013/0,296,165). In both cases, the synergistic mixture affords an enhanced glassy structure without the need for foaming or boiling under vacuum to facilitate effective drying.

The development of novel formulations is, however, a challenging task and not all formulation are effective for a given bacteria (Youg et al., Biotechnol Bioeng., 2006 Sep. 5; 95(1):76-83).

In light of the above, there is a need to provide improved preservation and storage conditions for E. coli bacteria.

### SUMMARY

The present disclosure relates broadly to a viable Escherichia coli (E. coli) embedded in a matrix, wherein said matrix has a water activity (a_{w}) of ≤0.3, and wherein said matrix comprises a first polysaccharide which is a hydrocolloid-forming polysaccharide, a second polysaccharide which is different from the first polysaccharide, and a disaccharide which includes sucrose, trehalose, or a combination thereof.

The present disclosure also relates broadly to a composition for forming a matrix, said composition comprising a first polysaccharide which is a hydrocolloid-forming polysaccharide, a second polysaccharide which is different from the first polysaccharide, and a disaccharide which includes sucrose, trehalose, or a combination thereof, and an Escherichia coli (E. coli).

The present disclosure also relates broadly to a method for providing a particulate comprising viable Escherichia coli (E. coli).

The present disclosure also relates broadly to a matrix comprising viable Escherichia coli (E. coli), wherein said matrix has a water activity (a_{w}) of ≤0.3, and wherein said matrix comprises a first polysaccharide which is a hydrocolloid-forming polysaccharide, a second polysaccharide which is different from the first polysaccharide, and a disaccharide which includes sucrose, trehalose, or a combination thereof.

All features of embodiments which are described in this disclosure and are not mutually exclusive can be combined with one another. Elements of one embodiment can be utilized in the other embodiments without further mention. Other aspects and features of the present invention will become apparent to those ordinarily skilled in the art upon review of the following description of specific embodiments in conjunction with the accompanying Figures.

### BRIEF DESCRIPTION OF THE DRAWINGS

A detailed description of specific embodiments is provided herein below with reference to the accompanying drawings in which:

FIG. 1 shows a non-limiting flow diagram for preparing a bacteria culture in accordance with an embodiment of the present disclosure.

FIG. 2 shows a non-limiting flow diagram for drying beads with embedded E. coli in accordance with an embodiment of the present disclosure.

FIG. 3 shows a non-limiting bar graph that depicts the effect of preservation solutions S1, S2, S3 and S4 on bacterial viability following air-drying in accordance with an embodiment of the present disclosure.

FIG. 4 shows a non-limiting bar graph that depicts the effect of preservation solutions S1, S5, S6 and S7 on bacterial viability following air-drying in accordance with an embodiment of the present disclosure.

FIG. 5 shows a non-limiting bar graph that depicts the effect of preservation solutions S1, S0, S8 and S9 on bacterial viability following air-drying in accordance with an embodiment of the present disclosure.

FIG. 6 shows a non-limiting bar graph that depicts the effect of preservation solutions S1, S10, S11 and S12 on bacterial viability following air-drying in accordance with an embodiment of the present disclosure.

FIG. 7 shows a non-limiting bar graph that depicts the effect of preservation solutions S1, S13, S14 and S15 on bacterial viability following air-drying in accordance with an embodiment of the present disclosure.

FIG. 8 shows a non-limiting bar graph that depicts the effect of preservation solutions S1, S16, S17 and S18 on bacterial viability following air-drying in accordance with an embodiment of the present disclosure.

FIG. 9 shows a non-limiting bar graph that depicts the effect of preservation solutions S1 and S19 on bacterial viability following air-drying in accordance with an embodiment of the present disclosure.

FIG. 10 shows the raw data regarding FIGS. 3 to 9.

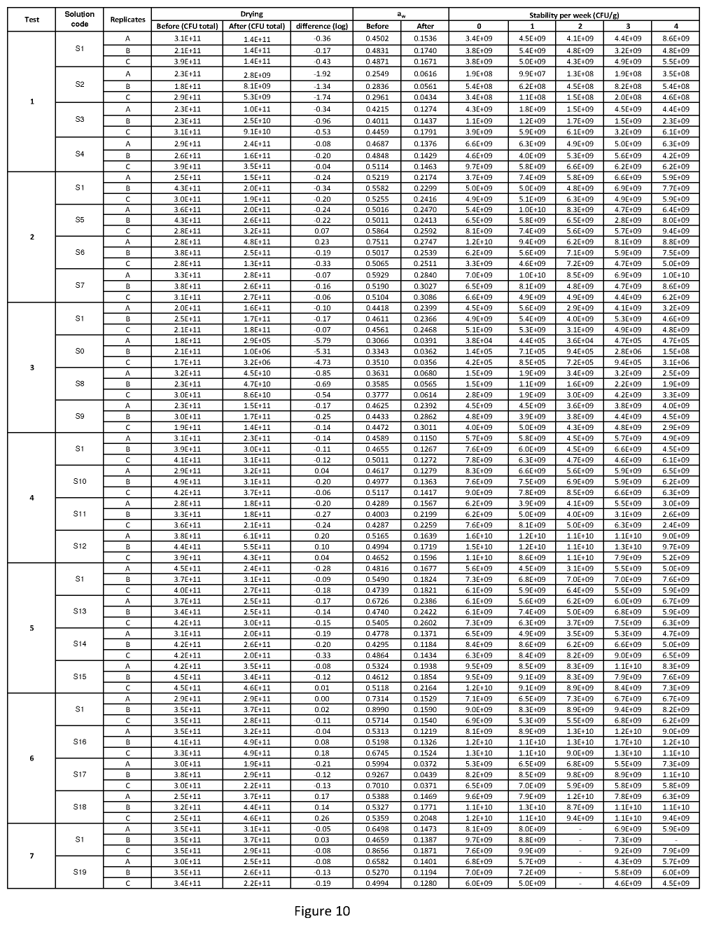

In the drawings, embodiments are illustrated by way of example. It is to be expressly understood that the description and drawings are only for the purpose of illustrating certain embodiments and are an aid for understanding. The scope of the claims should not be limited by the embodiments set forth in the present disclosure, but should be given the broadest interpretation consistent with the description as a whole.

### DETAILED DESCRIPTION OF EMBODIMENTS

Specific examples will now be described to illustrate the manner in which the principles of the present disclosure may be put into practice.

The herein described E. coli bacteria are viable bacteria, in other words, while the bacteria embedded in a dry matrix can be considered as being in a non-active state, these bacteria can be restored to an active state upon exposing the matrix to moisture.

The herein described E. coli bacteria comprise any recombinant or wild E. coli strain, or any mixtures thereof. In one embodiment, the E. coli is a non-pathogenic strain. In one embodiment, the non-pathogenic E. coli strain is the strain deposited at the International Depository Authority of Canada (IDAC) on Jan. 21, 2005 under accession number IDAC 210105-01, or the strain deposited at the International Depositary Authority of Canada (IDAC) on Jun. 20, 2013 and attributed accession number 200613-01, or a combination thereof.

The herein described matrix comprises a hydrocolloid-forming polysaccharide. Several polysaccharides are suitable for use as described herein, alone or in any combination thereof.

High amylose starch is a polysaccharide capable of forming firm gel after hydrating the starch granules in boiling water, dispersing the granules with the aid of high shear mixer and then cooling the solution to about 0-10° C. The firmness and strength of the gel depend on the concentration of the starch in the solution, with a maximal workable concentration of up to 10% w/v. The sliced starch gel matrix is also capable of retaining the live bacteria in the preservation mixture, and since it is mostly non-digestible by intestinal or gastric juices, the bacteria are protected from gastric destruction while within the starch matrix. The controlled release mechanism is provided by the fact that high amylose starch is readily digestible by the gut microflora at which time the delivered live bacteria are then released in their intact form.

Pectin is another suitable polysaccharide that performs very similar to high amylose starch. Pectin has an additional advantage since the strength of the pectin gel matrix can be further increased by the addition of divalent cations such as Ca²⁺ that forms bridges between carboxyl groups of the sugar polymers.

Alginate is another suitable polysaccharide that can form a firm gel matrix by cross-linking with divalent cations. The alginate can be hardened into a firm gel matrix by internally cross-linking the alginate polysaccharides with a dication, e.g. Ca²⁺, for example by extruding the alginate in the form of thin threads, strings, or substantially spherical beads into a Ca²⁺ bath. The alginate hardens upon interaction with Ca²⁺. An alternative method of preparation of the matrix is to spray atomize the mixture into a bath containing Ca²⁺.

In one embodiment, the hydrocolloid-forming polysaccharide is present in the matrix in percent by weight of total dry matter at a value of from 0.1% to 20%. In one embodiment, the hydrocolloid-forming polysaccharide is present in the matrix in percent by weight of total dry matter at a value of from 0.1% to 19%, or from 0.1% to 18%, or from 0.1% to 17%, or from 0.1% to 16%, or from 0.1% to 15%, or from 0.1% to 14%, or from 0.1% to 13%, or from 0.1% to 12%, or from 1% to 12%, including any value therein.

The herein described matrix further comprises a disaccharide and a polysaccharide. The present disclosure discloses several concentrations and proportions suitable for inclusion in the matrix. In one embodiment, a suitable ratio of disaccharide/polysaccharide in wt. %/wt. % is of less than 10 or more preferably of less than 5. In one embodiment, the ratio of disaccharide/polysaccharide in wt. %/wt. % is of about 1.

In one embodiment, the disaccharide is present in the matrix in percent by weight of total dry matter at a value of from 0.1% to 90%, or from 0.1% to 75%, or from 0.1% to 50%, or from 0.1% to 35%, or from 0.1% to 20%, or from 0.1% to 15%, or from 0.1% to 10%, including any value therein.

In one non-limiting embodiment, the disaccharide includes sucrose.

In a further non-limiting embodiment, the disaccharide includes trehalose.

In one non-limiting embodiment, the polysaccharide includes maltodextrine.

In a further non-limiting embodiment, the polysaccharide includes dextran.

In a further non-limiting embodiment, the dextran has a molecular weight between 20 and 70 kDa.

In one embodiment, the matrix further includes a salt of L-glutamic acid. In one non-limiting embodiment, the salt is a sodium salt of L-glutamic acid.

The herein described matrix has a water activity (“a_{w}”) which is of 0.04≤a_{W}≤0.3, for example 0.04≤a_{w}≤2.5, 0.04≤a_{w}≤2.0, 0.04≤a_{w}≤1.5, and the like. “Water activity” or “a_{w}” in the context of the present disclosure, refers to the availability of water and represents the energy status of the water in a system. Water activity may be measured according to materials and procedures known in the art, for example, using an Aqualab Water Activity Meter 4TE (Decagon Devices, Inc., U.S.A.).

There is also provided a composition for forming a matrix, the composition comprising a first hydrocolloid-forming polysaccharide, a second polysaccharide which is different from the first polysaccharide, and a disaccharide which includes sucrose, trehalose, or a combination thereof and an Escherichia coli (E. coli).

There is also provided a method for providing a particulate comprising viable Escherichia coli (E. coli), the method comprising providing particles comprising a first hydrocolloid-forming polysaccharide, a second polysaccharide which is different from the first polysaccharide, and a disaccharide which includes sucrose, trehalose, or a combination thereof and E. coli and drying said particles to water water activity (a_{w})≤0.3.

In one non-limiting embodiment, the viable E. coli sustains an a_{w} fold reduction in the particles of at least 0.4, or at least 0.5, or at least 0.6, or at least 0.7.

### EXAMPLES

In each of the following examples, three preservation solutions were tested along with preservation solution S1. The tests were performed in triplicates and one standard deviation was calculated according to the following formula:

$$SD = \sqrt{\frac{\sum {( x - \overline{x} )}^{2}}{n}}$$

with n: number of samples and \tilde{x}: mean of sample population.

In each of the following examples, bacterial viability was assessed by measuring the number of colony-forming units (CFU) according to protocols known in the art.

The preservation solutions used in the following examples are shown in Table 1.

TABLE 1

| | | | | Ratio |
|---|---|---|---|---|
| preser- | | | salt of | polysaccharide/ |
| vation | | | L-glutamic | disaccharide/salt |
| solution | Polysaccharide | Disaccharide | acid | of organic acid |
| S0 | x ¹ | x | x | N/A ² |
| S1 | dextran 40 | sucrose | yes | 5:7:1 |
| S2 | dextran 40 | x | x | N/A |
| | (5 wt %) | | | |
| S3 | x | Sucrose | x | N/A |
| | | (7 wt %) | | |
| S4 | dextran 40 | trehalose | yes | 5:7:1 |
| S5 | dextran 20 | sucrose | yes | 5:7:1 |
| S6 | dextran 70 | sucrose | yes | 5:7:1 |
| S7 | maltodextrine | sucrose | yes | 5:7:1 |
| S8 | dextran 40 | sucrose | yes | 10:1:1 |
| S9 | dextran 40 | sucrose | yes | 1:10:1 |
| S10 | dextran 40 | sucrose | yes | 5:7:1 |
| | (different | | | |
| | manufacturer) | | | |
| S11 | x | sucrose | yes | 7:1 |
| S12 | dextran 70 | trehalose | yes | 5:7:1 |
| S13 | dextran 40 | sucrose | x | 5:7 |
| S14 | dextran 40 | sucrose | yes | 5:3:1 |
| S15 | dextran 40 | sucrose | yes | 5:5:1 |
| S16 | maltodextrin | trehalose | yes | 5:7:1 |
| S17 | maltodextrin | trehalose | yes | 10:1:1 |
| S18 | maltodextrin | trehalose | yes | 1:10:1 |
| S19 | dextran 40 | maltose | yes | 5:7:1 |

¹ x means absent
² N/A means not applicable

### 1. Example 1

a. E. coli Culture

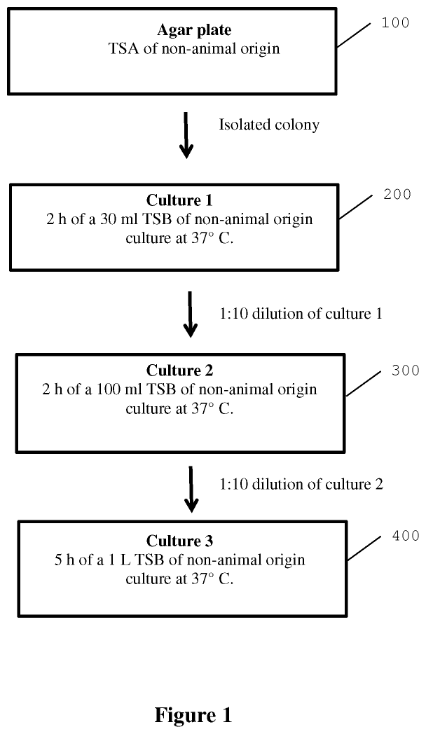

With reference to FIG. 1, an E. coli strain was cultivated in a first step 100 on Tryptic Soy Agar of non-animal origin. Six (6) isolated colonies were then used to cultivate the E. coli strain in a second step 200 for 2 hours at 37° C. and agitation at 200 rpm in 30 mL of Tryptic Soy Broth (TSB) of non-animal origin (for 1 L of TSB: 20 g of Soy Peptone A3 SC—(Organotechnie), 2.5 g anhydrous dextrose USP—(J. T. Baker), 5 g sodium chloride USP—(J. T. Baker), and 2.5 g dibasic potassium phosphate USP—(Fisher Chemical)). The resulting Culture 1 was diluted by a factor of 10 in TSB and was then used to cultivate the E. coli strain in a third step 300 for 2 hours at 37° C. and agitation at 200 rpm in 100 mL of TSB of non-animal origin. The resulting Culture 2 was diluted by a factor of 10 in TSB and was then used to cultivate the E. coli strain in a fourth step 400 for 5 hours at 37° C. and agitation at 200 rpm in 1 L of TSB of non-animal origin. The resulting Culture 3 was then used to embed E. coli in matrix. Variations and refinements to the culture protocol herein described are possible and will become apparent to persons skilled in the art in light of the present teachings. For example, the non-pathogenic E. coli may also be cultivated in anaerobic conditions according to protocols known in the art (Son & Taylor, Curr. Protoc. Microbiol., 2012, 27:5A.4.1-5A.4.9). In preparing the beads of the subsequent examples, the non-pathogenic E. coli strain deposited at the International Depository Authority of Canada (IDAC) on Jan. 21, 2005 under accession number IDAC 210105-01 may be selected.

b. Matrix Preparation

Bacto™ peptone (1.5 g, BD, Mississauga, Canada) was mixed with 1.5 L of heated water to obtain a mixture. Alginate (30 g Grindsted®, DuPont™ Danisco®, Mississauga, Canada) was slowly added to the mixture while mixing with a magnetic bar at 360 rpm. Complete solubilisation of alginate was obtained in about 3 h to obtain a 2% alginate (m/v) solution. The solution including the magnetic bar was then autoclaved under standard conditions. Variations and refinements to the matrix preparation protocol herein described are possible and will become apparent to persons skilled in the art in light of the present teachings.

c. Embedding E. coli in Matrix

The following was added, in order and while mixing with the magnetic bar, to the autoclaved matrix solution to obtain a slurry: 1 L of TSB of non-animal origin and, with reference to FIG. 1, 0.5 L of the resulting Culture 3 of E. coli in 1 L of TSB of non-animal origin. The slurry was extruded into a polymerization bath (300 mM CaCl₂), 0.1 wt./v. % Bacto™ tryptone, 0.1 wt./v. % Bacto™ peptone, and 0.05 wt./v. % g Bacto™ yeast extract in water) to form beads using a 9 exit syringe system adapted from the Thermo Scientific™ Reacti-Vap™ Evaporators. The bath was gently stirred while injecting the slurry. The matrix beads were allowed to cross-link for about 30 minutes, and the resulting hardened beads were then harvested. Variations and refinements to the embedding protocol herein described are possible and will become apparent to persons skilled in the art in light of the present teachings.

d. Drying and Testing of Embedded E. coli

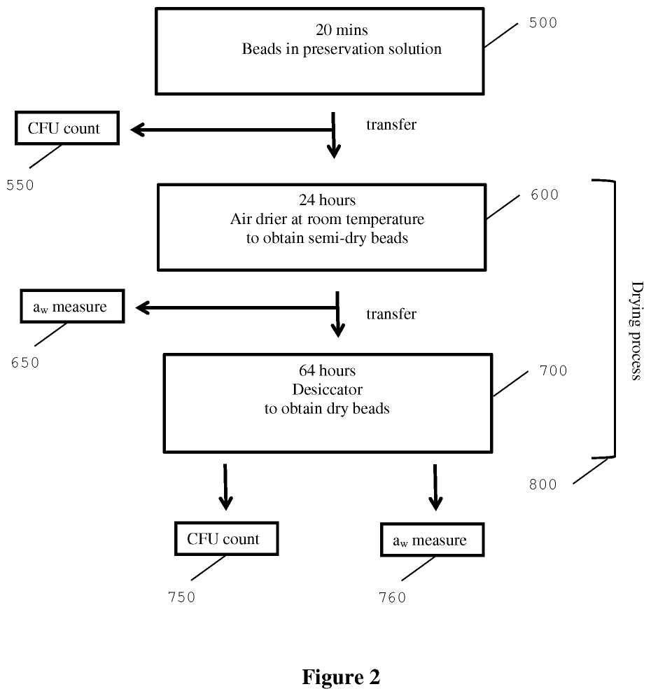

For each preservation solution the drying and testing was performed at least in triplicates. With reference to FIG. 2, in a first step 500 the beads with embedded E. coli in the matrix were placed in a preservation solution S1, a preservation solution S2, a preservation solution S3 or a preservation solution S4 with gentle stirring for about 20 minutes. In each case, a determination of total CFU 550 was performed after soaking in the preservation solution. In a second step 600 the beads were then placed on a tray dryer in an air dryer at room temperature for about 24 h to obtain semi-dry beads. In each case, a measurement of water activity a_{w} 650 was performed on the semi-dry beads using an Aqualab Water Activity Meter 4TE (Decagon Devices, Inc., U.S.A.). In a third step 700 the semi-dry beads were then placed in a desiccator for about 64 h, in which dry and filtered air was blown. In accordance with an embodiment of the present disclosure, the drying process 800 includes at least two steps: a step 600 which includes placing beads in an air dryer for 24 hours at room temperature and to obtain semi-dry beads and a step 700 which includes placing the semi-dry beads in a desiccator for 64 hours to obtain dry beads. In each case, a determination of total CFU 750 and a measurement of water a_{w} 760 were performed on the dry beads. Dry beads having a water activity a_{w} of ≤0.3 were obtained.

In each case, and with reference to FIG. 2, the a_{w} fold reduction was calculated according to the following:

$${a}_{w} fold reduction = \frac{650 - 760}{650}$$

In each case, and with reference to FIG. 2, viability loss was calculated according to the following:

CFU loss=log₁₀(550)−log₁₀(750)

In each case, an average viability loss and normalized average viability loss relative to the results obtained with preservation solution S1 was calculated.

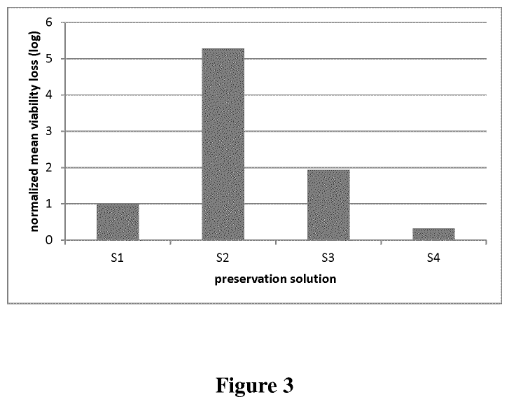

The results are shown in FIG. 3. Preservation solution S4 showed a normalized average viability loss of 0.32 while sustaining a water activity of 0.142±0.004.

A compilation of the results of Example 1 is set forth in Tables 2 and 3. These results demonstrate that the elements of preservation solution S4 provided a significant effect to the viability of the E. coli embedded in the dried matrix and its resistance to the drying process 700.

TABLE 2

| | | step 750 | | |
|---|---|---|---|---|
| | | | average | Normalized |
| | step 550 | | CFU loss | CFU loss |
| Sample | average CFU | average CFU | (log₁₀) | (log₁₀) |
| S1 | 3 × 10¹¹ ± 9 × 10¹⁰ | 1.4 × 10¹¹ ± 4.3 × 10⁹ | 0.32 ± 0.14 | 1 |
| S2 | 2.3 × 10¹¹ ± 5.5 × 10¹⁰ | 5.4 × 10⁹ ± 2.7 × 10⁹ | 1.66 ± 0.3 | 5.18 |
| S3 | 2.6 × 10¹¹ ± 4.9 × 10¹⁰ | 7.4 × 10¹⁰ ± 4.3 × 10¹⁰ | 0.61 ± 0.32 | 1.90 |
| S4 | 3.1 × 10¹¹ ± 7 × 10¹⁰ | 2.5 × 10¹¹ ± 9.7 × 10¹⁰ | 0.11 ± 0.08 | 0.34 |

TABLE 3

step 760

| | step 650 | | a_{w} fold |
|---|---|---|---|
| Sample | average a_{w} | average a_{w} | reduction |
| S1 | 0.473 ± 0.020 | 0.165 ± 0.010 | 0.65 |
| S2 | 0.278 ± 0.021 | 0.054 ± 0.009 | 0.81 |
| S3 | 0.423 ± 0.022 | 0.150 ± 0.026 | 0.64 |
| S4 | 0.488 ± 0.022 | 0.142 ± 0.004 | 0.71 |

e. Incorporating Dried Embedded E. coli into a Feed (“Pelleting”)

Protocol for incorporating dried matrix into a feed, for example in the form of a feed additive are known in the art. An illustrative example of doing such can be done, e.g., by incorporating 500 g to 1000 g of dried matrix beads into a ton of feed. If desired, the feed can also include inactivated yeast product in suitable amounts. For instance, the dried matrix beads comprising the embedded E. coli are mixed in a homogenization tank with all other ingredients. Preferably, the mixture is continuously mixed during the pelleting process. The mixed material is then pumped towards an extruder. Steam is then applied on the mixed material that is about to enter the extruder (i.e., hence, the temperature of the mixture increases at this stage). Suitable pressure is then applied on the mixture during its passage inside the extruder (pressure and temperature increase, point where highest temperature reached, around 75° C.). The formed pellets are then expelled out of the extruder into a cooling tank (rapid temperature drops to 30-40° C. followed by another cool down, to reach ambient temperature). Pelleted feed including the feed additive (matrix comprising embedded E. coli) can then be stored, for example in bags/containers. Variations and refinements to the pelleting protocol herein described are possible and will become apparent to persons skilled in the art in light of the present teachings.

### 2. Example 2

For each preservation solution the drying and testing was performed at least in triplicates. With reference to FIG. 2, in a first step 500 beads prepared as in Example 1 were placed in a preservation solution S1, a preservation solution S5, a preservation solution S6 or a preservation solution S7 with gentle stirring for about 20 minutes. In each case, a determination of total CFU 550 was performed after soaking in the preservation solution. In a second step 600 the beads were then placed on a tray dryer in an air dryer at room temperature for about 24 h to obtain semi-dry beads. In each case, a measurement of water activity a_{w} 650 was performed on the semi-dry beads using an Aqualab Water Activity Meter 4TE (Decagon Devices, Inc., U.S.A.). In a third step 700 the semi-dry beads were then placed in a desiccator for about 64 h, in which dry and filtered air was blown. In accordance with an embodiment of the present disclosure, the drying process 800 includes at least two steps: a step 600 which includes placing beads in an air dryer for 24 hours at room temperature and to obtain semi-dry beads and a step 700 which includes placing the semi-dry beads in a desiccator for 64 hours to obtain dry beads. In each case, a determination of total CFU 750 and a measurement of water a_{w} 760 were performed on the dry beads. Dry beads having a water activity a_{w} of ≤0.3 were obtained.

In each case, and with reference to FIG. 2, the a_{w} fold reduction was calculated according to the following:

$${a}_{w} fold reduction = \frac{650 - 760}{650}$$

In each case, and with reference to FIG. 2, viability loss was calculated according to the following:

CFU loss=log₁₀(550)−log₁₀(750)

In each case, an average viability loss and normalized average viability loss relative to the results obtained with preservation solution S1 was calculated.

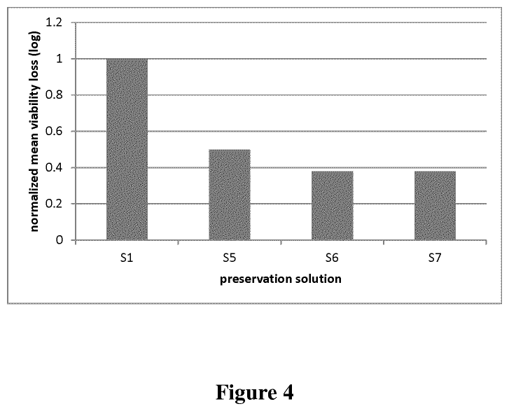

The results are shown in FIG. 4. Preservation solution S7 showed a normalized average viability loss of 0.38 while sustaining a water activity of 0.298±0.013.

A compilation of the results of Example 2 is set forth in Tables 4 and 5. These results demonstrate that the elements of preservation solution S7 provided a significant protective effect to the viability of the E. coli embedded in the dried matrix and its resistance to the drying process 700.

TABLE 4

| | | step 750 | | |
|---|---|---|---|---|
| | | | average | Normalized |
| | step 550 | | CFU loss | CFU loss |
| Sample | average CFU | average CFU | (log₁₀) | (log₁₀) |
| S1 | 3.3 × 10¹¹ ± 9.2 × 10¹⁰ | 1.8 × 10¹¹ ± 2.7 × 10¹⁰ | 0.26 ± 0.07 | 1 |
| S5 | 3.5 × 10¹¹ ± 7.7 × 10¹⁰ | 2.6 × 10¹¹ ± 6 × 10¹⁰ | 0.13 ± 0.17 | 0.5 |
| S6 | 3.1 × 10¹¹ ± 5.8 × 10¹⁰ | 2.8 × 10¹¹ ± 1.7 × 10¹¹ | 0.10 ± 0.29 | 0.38 |
| S7 | 3.4 × 10¹¹ ± 3.7 × 10¹⁰ | 2.7 × 10¹¹ ± 1 × 10¹⁰ | 0.10 ± 0.06 | 0.38 |

TABLE 5

step 760

| | step 650 | | a_{w} fold |
|---|---|---|---|
| Sample | average a_{w} | average a_{w} | reduction |
| S1 | 0.535 ± 0.020 | 0.230 ± 0.012 | 0.57 |
| S5 | 0.530 ± 0.049 | 0.249 ± 0.009 | 0.53 |
| S6 | 0.586 ± 0.143 | 0.260 ± 0.013 | 0.56 |
| S7 | 0.541 ± 0.045 | 0.298 ± 0.013 | 0.45 |

### 3. Example 3

For each preservation solution the drying and testing was performed at least in triplicates. With reference to FIG. 2, in a first step 500 beads prepared as in Example 1 were placed in either a preservation solution S1, a preservation solution S0, a preservation solution S8 or a preservation solution S9 with gentle stirring for about 20 minutes. In each case, a determination of total CFU 550 was performed after soaking in the preservation solution. In a second step 600 the beads were then placed on a tray dryer in an air dryer at room temperature for about 24 h to obtain semi-dry beads. In each case, a measurement of water activity a_{w} 650 was performed on the semi-dry beads using an Aqualab Water Activity Meter 4TE (Decagon Devices, Inc., U.S.A.). In a third step 700 the semi-dry beads were then placed in a desiccator for about 64 h, in which dry and filtered air was blown. In accordance with an embodiment of the present disclosure, the drying process 800 includes at least two steps: a step 600 which includes placing beads in an air dryer for 24 hours at room temperature and to obtain semi-dry beads and a step 700 which includes placing the semi-dry beads in a desiccator for 64 hours to obtain dry beads. In each case, a determination of total CFU 750 and a measurement of water a_{w} 760 were performed on the dry beads. Dry beads having a water activity a_{w} of ≤0.3 were obtained.

In each case, and with reference to FIG. 2, the a_{w} fold reduction was calculated according to the following:

$${a}_{w} fold reduction = \frac{650 - 760}{650}$$

In each case, and with reference to FIG. 2, viability loss was calculated according to the following:

CFU loss=log₁₀(550)−log₁₀(750)

In each case, an average viability loss and normalized average viability loss relative to the results obtained with preservation solution S1 was calculated.

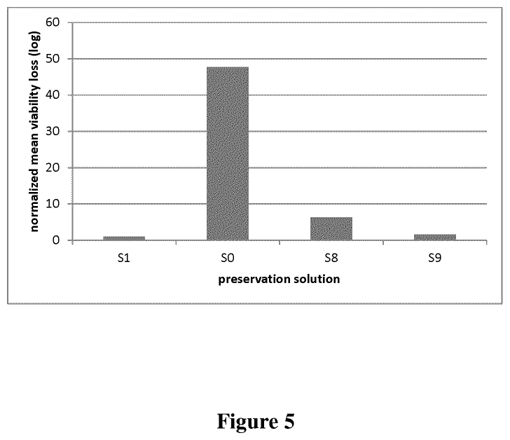

The results are shown in FIG. 5.

A compilation of the results of Example 3 is set forth in Tables 6 and 7.

TABLE 6

| | | step 750 | | |
|---|---|---|---|---|
| | | | average | Normalized |
| | step 550 | | CFU loss | CFU loss |
| Sample | average CFU | average CFU | (log₁₀) | (log₁₀) |
| S1 | 2.2 × 10¹¹ ± 2.9 × 10¹⁰ | 1.7 × 10¹¹ ± 9.3 × 10⁹ | 0.11 ± 0.05 | 1 |
| S0 | 1.9 × 10¹¹ ± 2 × 10¹⁰ | 1.5 × 10⁶ ± 1.5 × 10⁶ | 5.28 ± 0.53 | 47.7 |
| S8 | 2.8 × 10¹¹ ± 4.6 × 10¹⁰ | 5.9 × 10¹⁰ ± 2.3 × 10¹⁰ | 0.70 ± 0.15 | 6.28 |
| S9 | 2.4 × 10¹¹ ± 5.4 × 10¹⁰ | 1.5 × 10¹¹ ± 1.5 × 10¹⁰ | 0.18 ± 0.04 | 1.64 |

TABLE 7

step 760

| | step 650 | | a_{w} fold |
|---|---|---|---|
| Sample | average a_{w} | average a_{w} | reduction |
| S1 | 0.453 ± 0.010 | 0.241 ± 0.005 | 0.47 |
| S0 | 0.331 ± 0.022 | 0.037 ± 0.002 | 0.89 |
| S8 | 0.366 ± 0.010 | 0.062 ± 0.006 | 0.83 |
| S9 | 0.451 ± 0.010 | 0.275 ± 0.032 | 0.39 |

### 4. Example 4

For each preservation solution the drying and testing was performed at least in triplicates. With reference to FIG. 2, in a first step 500 beads prepared as in Example 1 were placed in either a preservation solution S1, a preservation solution S10, a preservation solution S11 or a preservation solution S12 with gentle stirring for about 20 minutes. In each case, a determination of total CFU 550 was performed after soaking in the preservation solution. In a second step 600 the beads were then placed on a tray dryer in an air dryer at room temperature for about 24 h to obtain semi-dry beads. In each case, a measurement of water activity a_{w} 650 was performed on the semi-dry beads using an Aqualab Water Activity Meter 4TE (Decagon Devices, Inc., U.S.A.). In a third step 700 the semi-dry beads were then placed in a desiccator for about 64 h, in which dry and filtered air was blown. In accordance with an embodiment of the present disclosure, the drying process 800 includes at least two steps: a step 600 which includes placing beads in an air dryer for 24 hours at room temperature and to obtain semi-dry beads and a step 700 which includes placing the semi-dry beads in a desiccator for 64 hours to obtain dry beads. In each case, a determination of total CFU 750 and a measurement of water a_{w} 760 were performed on the dry beads. Dry beads having a water activity a_{w} of ≤0.3 were obtained.

In each case, and with reference to FIG. 2, the a_{w} fold reduction was calculated according to the following:

$${a}_{w} fold reduction = \frac{650 - 760}{650}$$

In each case, and with reference to FIG. 2, viability loss was calculated according to the following:

CFU loss=log₁₀(550)−log₁₀(750)

In each case, an average viability loss and normalized average viability loss relative to the results obtained with preservation solution S1 was calculated.

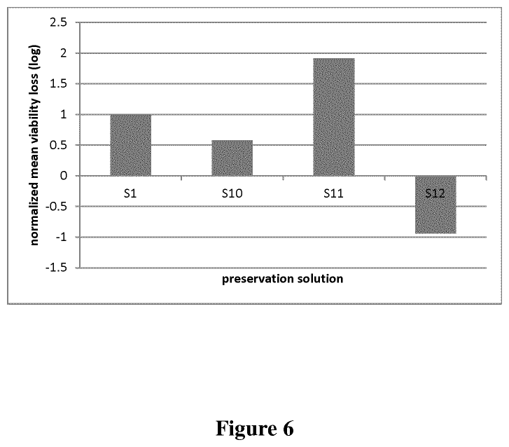

The results are shown in FIG. 6. Preservation solution S7 showed a normalized average viability loss of 0.58.

A compilation of the results of Example 4 is set forth in Tables 8 and 9.

TABLE 8

| | | step 750 | | |
|---|---|---|---|---|
| | | | average | Normalized |
| | step 550 | | CFU loss | CFU loss |
| Sample | average CFU | average CFU | (log₁₀₎ | (log₁₀) |
| S1 | 3.7 × 10¹¹ ± 5.2 × 10¹⁰ | 2.8 × 10¹¹ ± 4.46 × 10¹⁰ | 0.12 ± 0.01 | 1 |
| S10 | 4 × 10¹¹ ± 1 × 10¹¹ | 3.3 × 10¹¹ ± 3.11 × 10¹⁰ | 0.07 ± 0.12 | 0.58 |
| S11 | 3.3 × 10¹¹ ± 3.9 × 10¹⁰ | 1.9 × 10¹¹ ± 1.77 × 10¹⁰ | 0.24 ± 0.03 | 1.91 |
| S12 | 4 × 10¹¹ ± 2.9 × 10¹⁰ | 5.3 × 10¹¹ ± 9.27 × 10¹⁰ | −0.12 ± 0.08 | −0.94 |

TABLE 9

step 760

| | step 650 | | a_{w} fold |
|---|---|---|---|
| Sample | average a_{w} | average a_{w} | reduction |
| S1 | 0.475 ± 0.023 | 0.123 ± 0.007 | 0.74 |
| S10 | 0.490 ± 0.026 | 0.135 ± 0.007 | 0.72 |
| S11 | 0.419 ± 0.016 | 0.201 ± 0.038 | 0.52 |
| S12 | 0.494 ± 0.026 | 0.165 ± 0.006 | 0.66 |

### 5. Example 5

For each preservation solution the drying and testing was performed at least in triplicates. With reference to FIG. 2, in a first step 500 beads prepared as in Example 1 were placed in either a preservation solution S1, a preservation solution S13, a preservation solution S14 or a preservation solution S15 with gentle stirring for about 20 minutes. In each case, a determination of total CFU 550 was performed after soaking in the preservation solution. In a second step 600 the beads were then placed on a tray dryer in an air dryer at room temperature for about 24 h to obtain semi-dry beads. In each case, a measurement of water activity a_{w} 650 was performed on the semi-dry beads using an Aqualab Water Activity Meter 4TE (Decagon Devices, Inc., U.S.A.). In a third step 700 the semi-dry beads were then placed in a desiccator for about 64 h, in which dry and filtered air was blown. In accordance with an embodiment of the present disclosure, the drying process 800 includes at least two steps: a step 600 which includes placing beads in an air dryer for 24 hours at room temperature and to obtain semi-dry beads and a step 700 which includes placing the semi-dry beads in a desiccator for 64 hours to obtain dry beads. In each case, a determination of total CFU 750 and a measurement of water a_{w} 760 were performed on the dry beads. Dry beads having a water activity a_{w} of ≤0.3 were obtained.

In each case, and with reference to FIG. 2, the a_{w} fold reduction was calculated according to the following:

$${a}_{w} fold reduction = \frac{650 - 760}{650}$$

In each case, and with reference to FIG. 2, viability loss was calculated according to the following:

CFU loss=log₁₀(550)−log₁₀(750)

In each case, an average viability loss and normalized average viability loss relative to the results obtained with preservation solution S1 was calculated.

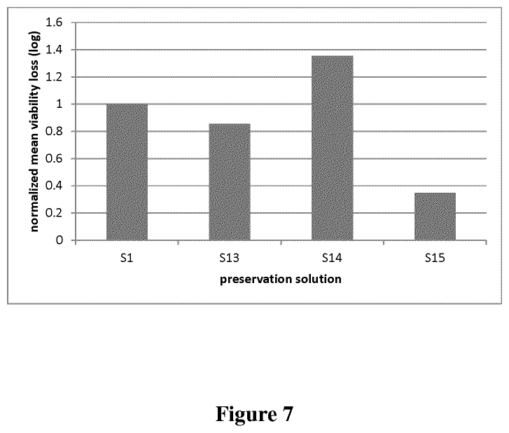

The results are shown in FIG. 7. Preservation solution S7 showed a normalized average viability loss of 0.35.

A compilation of the results of Example 5 is set forth in Tables 10 and 11.

TABLE 10

| | | step 750 | | |
|---|---|---|---|---|
| | | | average | Normalized |
| | step 550 | | CFU loss | CFU loss |
| Sample | average CFU | average CFU | (log₁₀) | (log₁₀) |
| S1 | 4.1 × 10¹¹ ± 3.8 × 10¹⁰ | 2.7 × 10¹¹ ± 3.44 × 10¹⁰ | 0.18 ± 0.10 | 1 |
| S13 | 3.8 × 10¹¹ ± 4.2 × 10¹⁰ | 2.6 × 10¹¹ ± 2.7 × 10¹⁰ | 0.15 ± 0.02 | 0.85 |
| S14 | 3.8 × 10¹¹ ± 6.1 × 10¹⁰ | 2.2 × 10¹¹ ± 3.55 × 10¹⁰ | 0.24 ± 0.08 | 1.35 |
| S15 | 4.4 × 10¹¹ ± 1.5 × 10¹⁰ | 3.8 × 10¹¹ ± 6.37 × 10¹⁰ | 0.06 ± 0.07 | 0.35 |

TABLE 11

step 760

| | step 650 | | a_{w} fold |
|---|---|---|---|
| Sample | average a_{w} | average a_{w} | reduction |
| S1 | 0.501 ± 0.041 | 0.177 ± 0.008 | 0.65 |
| S13 | 0.562 ± 0.101 | 0.247 ± 0.012 | 0.56 |
| S14 | 0.465 ± 0.031 | 0.133 ± 0.013 | 0.71 |
| S15 | 0.502 ± 0.037 | 0.198 ± 0.016 | 0.60 |

### 6. Example 6

For each preservation solution the drying and testing was performed at least in triplicates. With reference to FIG. 2, in a first step 500 beads prepared as in Example 1 were placed in either a preservation solution S1, a preservation solution S16, a preservation solution S17 or a preservation solution S18 with gentle stirring for about 20 minutes. In each case, a determination of total CFU 550 was performed after soaking in the preservation solution. In a second step 600 the beads were then placed on a tray dryer in an air dryer at room temperature for about 24 h to obtain semi-dry beads. In each case, a measurement of water activity a_{w} 650 was performed on the semi-dry beads using an Aqualab Water Activity Meter 4TE (Decagon Devices, Inc., U.S.A.). In a third step 700 the semi-dry beads were then placed in a desiccator for about 64 h, in which dry and filtered air was blown. In accordance with an embodiment of the present disclosure, the drying process 800 includes at least two steps: a step 600 which includes placing beads in an air dryer for 24 hours at room temperature and to obtain semi-dry beads and a step 700 which includes placing the semi-dry beads in a desiccator for 64 hours to obtain dry beads. In each case, a determination of total CFU 750 and a measurement of water a_{w} 760 were performed on the dry beads. Dry beads having a water activity a_{w} of ≤0.3 were obtained.

In each case, and with reference to FIG. 2, the a_{w} fold reduction was calculated according to the following:

$${a}_{w} fold reduction = \frac{650 - 760}{650}$$

In each case, and with reference to FIG. 2, viability loss was calculated according to the following:

CFU loss=log₁₀(550)−log₁₀(750)

In each case, an average viability loss and normalized average viability loss relative to the results obtained with preservation solution S1 was calculated.

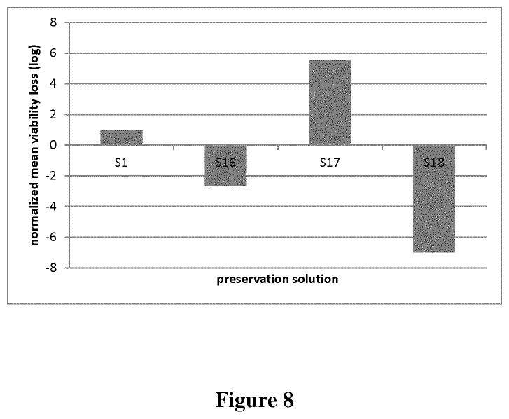

The results are shown in FIG. 8.

A compilation of the results of Example 6 is set forth in Tables 12 and 13.

TABLE 12

| | | step 750 | | |
|---|---|---|---|---|
| | | | average | |
| | step 550 | | CFU loss | Normalized CFU loss |
| Sample | average CFU | average CFU | (log₁₀) | (log₁₀) |
| S1 | 3.3 × 10¹¹ ± 3.4 × 10¹⁰ | 3.1 × 10¹¹ ± 4.92 × 10¹⁰ | 0.03 ± 0.07 | 1 |
| S16 | 3.6 × 10¹¹ ± 4.1 × 10¹⁰ | 4.4 × 10¹¹ ± 9.91 × 10¹⁰ | −0.07 ± 0.11 | −2.68 |
| S17 | 3.2 × 10¹¹ ± 4.5 × 10¹⁰ | 2.3 × 10¹¹ ± 5.05 × 10¹⁰ | 0.15 ± 0.05 | 5.57 |
| S18 | 2.7 × 10¹¹ ± 3.9 × 10¹⁰ | 4.2 × 10¹¹ ± 4.76 × 10¹⁰ | −0.19 ± 0.06 | −6.98 |

TABLE 13

step 760

| | step 650 | | a_{w} fold |
|---|---|---|---|
| Sample | average a_{w} | average a_{w} | reduction |
| S1 | 0.734 ± 0.164 | 0.155 ± 0.003 | 0.79 |
| S16 | 0.575 ± 0.862 | 0.136 ± 0.015 | 0.76 |
| S17 | 0.742 ± 0.167 | 0.039 ± 0.004 | 0.95 |
| S18 | 0.536 ± 0.003 | 0.176 ± 0.029 | 0.67 |

### 7. Example 7

For each preservation solution the drying and testing was performed at least in triplicates. With reference to FIG. 2, in a first step 500 beads prepared as in Example 1 were placed in either a preservation solution S1 or a preservation solution S19 with gentle stirring for about 20 minutes. In each case, a determination of total CFU 550 was performed after soaking in the preservation solution. In a second step 600 the beads were then placed on a tray dryer in an air dryer at room temperature for about 24 h to obtain semi-dry beads. In each case, a measurement of water activity a_{w} 650 was performed on the semi-dry beads using an Aqualab Water Activity Meter 4TE (Decagon Devices, Inc., U.S.A.). In a third step 700 the semi-dry beads were then placed in a desiccator for about 64 h, in which dry and filtered air was blown. In accordance with an embodiment of the present disclosure, the drying process 800 includes at least two steps: a step 600 which includes placing beads in an air dryer for 24 hours at room temperature and to obtain semi-dry beads and a step 700 which includes placing the semi-dry beads in a desiccator for 64 hours to obtain dry beads. In each case, a determination of total CFU 750 and a measurement of water a_{w} 760 were performed on the dry beads. Dry beads having a water activity a_{w} of ≤0.3 were obtained.

In each case, and with reference to FIG. 2, the a_{w} fold reduction was calculated according to the following:

$${a}_{w} fold reduction = \frac{650 - 760}{650}$$

In each case, and with reference to FIG. 2, viability loss was calculated according to the following:

CFU loss=log₁₀(550)−log₁₀(750)

In each case, an average viability loss and normalized average viability loss relative to the results obtained with preservation solution S1 was calculated.

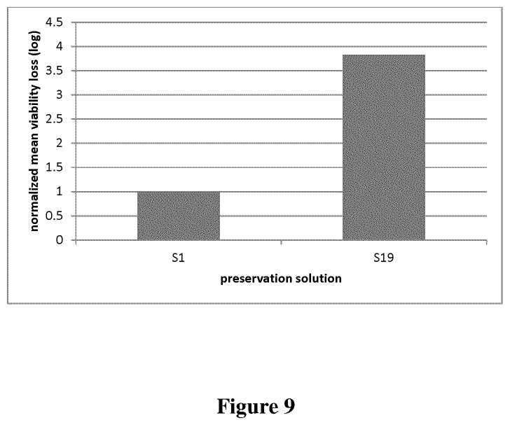

The results are shown in FIG. 9.

A compilation of the results of Example 7 is set forth in Tables 14 and 15.

TABLE 14

| | | step 750 | | |
|---|---|---|---|---|
| | | | average | |
| | step 550 | | CFU loss | Normalized CFU loss |
| Sample | average CFU | average CFU | (log₁₀) | (log₁₀) |
| S1 | 3.5 × 10¹¹ ± 4.4 × 10⁸ | 3.2 × 10¹¹ ± 4.13 × 10¹⁰ | 0.03 ± 0.05 | 1 |
| S19 | 3.3 × 10¹¹ ± 2.6 × 10¹⁰ | 2.4 × 10¹¹ ± 2.06 × 10¹⁰ | 0.13 ± 0.06 | 3.83 |

TABLE 15

step 760

| | step 650 | | a_{w} fold |
|---|---|---|---|
| Sample | average a_{w} | average a_{w} | reduction |
| S1 | 0.660 ± 0.200 | 0.158 ± 0.026 | 0.76 |
| S19 | 0.561 ± 0.085 | 0.129 ± 0.010 | 0.77 |

### 8. Example 8

For each preservation solution the drying and testing was performed at least in triplicates. Beads prepared as in Example 1 were placed in a preservation solution S1, a preservation solution S2, a preservation solution S3 and a preservation solution S4 with gentle stirring for about 20 minutes. The beads were then placed on a tray dryer in an air dryer at room temperature for about 24 h to obtain semi-dry beads. The semi-dry beads were then placed in a desiccator for about 64 h, in which dry and filtered air was blown. Dry beads having a water activity a_{w} of ≤0.3 were obtained.

In each case, the strain viability was tested over a period of four (4) weeks under storage conditions at 4° C. by measuring the CFU/g of the dried beads. The tests were performed at least in triplicates and one standard deviation was calculated.

The results of Example 8 are shown in Table 16 where all the preservation solutions tested afforded feed additive strain stability during 4 weeks when stored at 4° C.

TABLE 16

| Preservation | Difference CFU/g |
|---|---|
| solution | after 4 weeks (log) |
| S1 | 0.2 |
| S2 | 0.1 |
| S3 | 0.1 |
| S4 | 0 |

### 9. Example 9

For each preservation solution the drying and testing was performed at least in triplicates. Beads prepared as in Example 1 were placed in a preservation solution S1, a preservation solution S5, a preservation solution S6 and a preservation solution S7 with gentle stirring for about 20 minutes. The beads were then placed on a tray dryer in an air dryer at room temperature for about 24 h to obtain semi-dry beads. The semi-dry beads were then placed in a desiccator for about 64 h, in which dry and filtered air was blown. Dry beads having a water activity a_{w} of ≤0.3 were obtained.

In each case, the strain viability was tested over a period of four (4) weeks under storage conditions at 4° C. by measuring the CFU/g of the dried beads. The tests were performed at least in triplicates and one standard deviation was calculated.

The results of Example 9 are shown in Table 17 and all the preservation solutions tested afforded feed additive strain stability during 4 weeks when stored at 4° C.

TABLE 17

| Preservation | Difference CFU/g |
|---|---|
| solution | after 4 weeks (log) |
| S1 | 0.2 |
| S5 | 0.1 |
| S6 | 0 |
| S7 | 0.1 |

### 10. Example 10

For each preservation solution the drying and testing was performed at least in triplicates. Beads prepared as in Example 1 were placed in either a preservation solution S1, a preservation solution S0, a preservation solution S8 and a preservation solution S9 with gentle stirring for about 20 minutes. The beads were then placed on a tray dryer in an air dryer at room temperature for about 24 h to obtain semi-dry beads. The semi-dry beads were then placed in a desiccator for about 64 h, in which dry and filtered air was blown. Dry beads having a water activity a_{w} of ≤0.3 were obtained.

In each case, the strain viability was tested over a period of four (4) weeks under storage conditions at 4° C. by measuring the CFU/g of the dried beads. The tests were performed at least in triplicates and one standard deviation was calculated.

The results of Example 10 are shown in Table 18 and all the preservation solutions tested afforded feed additive strain stability during 4 weeks when stored at 4° C.

TABLE 18

| Preservation | Difference CFU/g |
|---|---|
| solution | after 4 weeks (log) |
| S1 | 0 |
| S0 | 2.4 |
| S8 | 0.1 |
| S9 | 0 |

### 11. Example 11

For each preservation solution the drying and testing was performed at least in triplicates. Beads prepared as in Example 1 were placed in either a preservation solution S1, a preservation solution S10, a preservation solution S11 and a preservation solution S12 with gentle stirring for about 20 minutes. The beads were then placed on a tray dryer in an air dryer at room temperature for about 24 h to obtain semi-dry beads. The semi-dry beads were then placed in a desiccator for about 64 h, in which dry and filtered air was blown. Dry beads having a water activity a_{w} of ≤0.3 were obtained.

In each case, the strain viability was tested over a period of four (4) weeks under storage conditions at 4° C. by measuring the CFU/g of the dried beads. The tests were performed at least in triplicates and one standard deviation was calculated.

The results of Example 11 are shown in Table 19 and all the preservation solutions tested afforded feed additive strain stability during 4 weeks when stored at 4° C.

TABLE 19

| Preservation | Difference CFU/g |
|---|---|
| solution | after 4 weeks (log) |
| S1 | 0.1 |
| S10 | 0.1 |
| S11 | 0.4 |
| S12 | 0.2 |

### 12. Example 12

For each preservation solution the drying and testing was performed at least in triplicates. Beads prepared as in Example 1 were placed in either a preservation solution S1, a preservation solution S13, a preservation solution S14 and a preservation solution S15 with gentle stirring for about 20 minutes. The beads were then placed on a tray dryer in an air dryer at room temperature for about 24 h to obtain semi-dry beads. The semi-dry beads were then placed in a desiccator for about 64 h, in which dry and filtered air was blown. Dry beads having a water activity a_{w} of ≤0.3 were obtained.

In each case, the strain viability was tested over a period of four (4) weeks under storage conditions at 4° C. by measuring the CFU/g of the dried beads. The tests were performed at least in triplicates and one standard deviation was calculated.

The results of Example 12 are shown in Table 20 and all the preservation solutions tested afforded feed additive strain stability during 4 weeks when stored at 4° C.

TABLE 20

| Preservation | Difference CFU/g |
|---|---|
| solution | after 4 weeks (log) |
| S1 | 0 |
| S13 | 0 |
| S14 | 0.1 |
| S15 | 0.1 |

### 13. Example 13

For each preservation solution the drying and testing was performed at least in triplicates. Beads prepared as in Example 1 were placed in either a preservation solution S1, a preservation solution S16, a preservation solution S17 and a preservation solution S18 with gentle stirring for about 20 minutes. The beads were then placed on a tray dryer in an air dryer at room temperature for about 24 h to obtain semi-dry beads. The semi-dry beads were then placed in a desiccator for about 64 h, in which dry and filtered air was blown. Dry beads having a water activity a_{w} of ≤0.3 were obtained.

In each case, the strain viability was tested over a period of four (4) weeks under storage conditions at 4° C. by measuring the CFU/g of the dried beads. The tests were performed at least in triplicates and one standard deviation was calculated.

The results of Example 13 are shown in Table 21 and all the preservation solutions tested afforded feed additive strain stability during 4 weeks when stored at 4° C.

TABLE 21

| Preservation | Difference CFU/g |
|---|---|
| solution | after 4 weeks (log) |
| S1 | 0 |
| S16 | 0 |
| S17 | 0 |
| S18 | 0.1 |

### 14. Example 14

For each preservation solution the drying and testing was performed at least in triplicates. Beads prepared as in Example 1 were placed in either a preservation solution S1 and a preservation solution S19 with gentle stirring for about 20 minutes. The beads were then placed on a tray dryer in an air dryer at room temperature for about 24 h to obtain semi-dry beads. The semi-dry beads were then placed in a desiccator for about 64 h, in which dry and filtered air was blown. Dry beads having a water activity a_{w} of ≤0.3 were obtained.

In each case, the strain viability was tested over a period of four (4) weeks under storage conditions at 4° C. by measuring the CFU/g of the dried beads. The tests were performed at least in triplicates and one standard deviation was calculated.

The results are shown in Table 22 and all the preservation solutions tested afforded feed additive strain stability during 4 weeks when stored at 4° C.

TABLE 22

| Preservation | Difference CFU/g |
|---|---|
| solution | after 4 weeks (log) |
| S1 | 0.1 |
| S19 | 0.1 |

In brief, the present inventor has surprisingly and unexpectedly observed that a matrix comprising embedded viable E. coli as described herein was capable of preserving viability of sufficient bacteria CFU over a given period of time, e.g. 4 weeks, for a commercial use thereof. For example, the matrix was successfully incorporated into a pelleted animal feed such that the animal feed could be stored/transported/handled and eventually administered to an animal while retaining sufficient viable CFU/g of animal feed to provide the beneficial effect normally associated with the bacteria.

Note that titles or subtitles may be used throughout the present disclosure for convenience of a reader, but in no way these should limit the scope of the invention. Moreover, certain theories may be proposed and disclosed herein; however, in no way they, whether they are right or wrong, should limit the scope of the invention so long as the invention is practiced according to the present disclosure without regard for any particular theory or scheme of action.

All references cited throughout the specification are hereby incorporated by reference in their entirety for all purposes.

It will be understood by those of skill in the art that throughout the present specification, the term “a” used before a term encompasses embodiments containing one or more to what the term refers. It will also be understood by those of skill in the art that throughout the present specification, the term “comprising”, which is synonymous with “including,” “containing,” or “characterized by,” is inclusive or open-ended and does not exclude additional, un-recited elements or method steps.

Unless otherwise defined, all technical and scientific terms used herein have the same meaning as commonly understood by one of ordinary skill in the art to which this invention pertains. In the case of conflict, the present document, including definitions will control.

As used in the present disclosure, the terms “around”, “about” or “approximately” shall generally mean within the error margin generally accepted in the art. Hence, numerical quantities given herein generally include such error margin such that the terms “around”, “about” or “approximately” can be inferred if not expressly stated.

Although the present disclosure has described in considerable detail certain embodiments, variations and refinements are possible and will become apparent to persons skilled in the art in light of the present teachings.

1. A particle comprising:
a first composition including a first polysaccharide in the form of a matrix and a viable Escherichia coli (E. coli), wherein the viable E. coli is embedded in the matrix, and wherein the first polysaccharide is a hydrocolloid-forming polysaccharide, and
a second composition free of the first polysaccharide, the second composition including a second polysaccharide and a disaccharide,
wherein the particle has a water activity (a_{w})≤0.3.

2. The particle of claim 1, wherein the first polysaccharide includes alginate.

3. The particle of claim 1, wherein 0.04≤a_{w}≤0.3.

4. The particle of claim 1, wherein said second composition comprises a ratio disaccharide/second polysaccharide of less than 10, wherein the ratio is wt. %/wt. %.

5. The particle of claim 4, wherein the ratio is of less than 5.

6. The particle of claim 4, wherein the ratio is of about 1.

7. The particle of claim 1, wherein said second composition further comprises a salt of L-glutamic acid.

8. The particle of claim 7, wherein said salt is sodium salt of L-glutamic acid.

9. The particle of claim 1, wherein said second polysaccharide includes maltodextrin.

10. The particle of claim 1, wherein said second polysaccharide includes dextran.

11. The particle of claim 1, wherein said E. coli includes a non-pathogenic E. coli.

12. The particle of claim 1, wherein said non-pathogenic E. coli includes the strain deposited at the International Depository Authority of Canada (IDAC) on Jan. 21, 2005, under accession number IDAC 210105-01.

13. The particle of claim 1, wherein said non-pathogenic E. coli includes the strain deposited at the International Depository Authority of Canada (IDAC) on Jun. 20, 2013, and attributed accession number 200613-01.

14. The particle of claim 1, wherein said hydrocolloid-forming polysaccharide is present in the matrix in percent by weight of total dry matter at a value of from 0.1% to 20%.

15. The particle of claim 1, wherein said hydrocolloid-forming polysaccharide is present in the matrix in percent by weight of total dry matter at a value of from 1 to 12%.

16. The particle of claim 1, wherein the first composition forms a core and the second composition is deposited on the core.

17. The particle of claim 1, wherein said second polysaccharide is dextran and said disaccharide is sucrose, present in a ration dextran:sucrose of from 10:1 to 1:10.

18. A particle comprising:
a first composition including a first polysaccharide in the form of a matrix and a viable Escherichia coli (E. coli), wherein the viable E. coli is embedded in the matrix, and wherein the first polysaccharide is a hydrocolloid-forming polysaccharide, and
a second composition being different from the first composition, the second composition including a second polysaccharide and a disaccharide,
wherein the particle has a water activity (a_{w})≤0.3.

19. The particle of claim 18, wherein the first polysaccharide includes alginate.

20. The particle of claim 18, wherein 0.04≤a_{W}≤0.3.

21. The particle of claim 18, wherein said second composition comprises a ratio disaccharide/second polysaccharide of less than 10, wherein the ratio is wt. %/wt. %.

22. The particle of claim 21, wherein the ratio is of less than 5.

23. The particle of claim 21, wherein the ratio is of about 1.

24. The particle of claim 18, wherein said second composition further comprises a salt of L-glutamic acid.

25. The particle of claim 24, wherein said salt is sodium salt of L-glutamic acid.

26. The particle of claim 18, wherein said second polysaccharide includes maltodextrin.

27. The particle of claim 18, wherein said second polysaccharide includes dextran.

28. The particle of claim 18, wherein said E. coli includes a non-pathogenic E. coli.

29. The particle of claim 18, wherein said non-pathogenic E. coli includes the strain deposited at the International Depository Authority of Canada (IDAC) on Jan. 21, 2005, under accession number IDAC 210105-01.

30. The particle of claim 18, wherein said non-pathogenic E. coli includes the strain deposited at the International Depository Authority of Canada (IDAC) on Jun. 20, 2013, and attributed accession number 200613-01.

31. The particle of claim 18, wherein said hydrocolloid-forming polysaccharide is present in the matrix in percent by weight of total dry matter at a value of from 0.1% to 20%.

32. The particle of claim 18, wherein said hydrocolloid-forming polysaccharide is present in the matrix in percent by weight of total dry matter at a value of from 1 to 12%.

33. The particle of claim 18, wherein the first composition forms a core and the second composition is deposited on the core.

34. The particle of claim 18, wherein said second polysaccharide is dextran and said disaccharide is sucrose, present in a ratio of dextran:sucrose of from 10:1 to 1:10.
